(19) United States
(12) Patent Application Publication
(10) Pub. No.: US 2023/0248875 A1
(43) Pub. Date: Aug. 10, 2023

(54) SYSTEM AND METHOD FOR KILLING MICROORGANISMS

(71) Applicant: Spencer W. HESS, Bushnell, FL (US)

(72) Inventor: Spencer W. HESS, Bushnell, FL (US)

(21) Appl. No.: 18/014,891

(22) PCT Filed: Jul. 6, 2021
(86) PCT No.: PCT/US2021/040425

§ 371 Date: Jan. 6, 2023

Related U.S. Application Data
(60) Provisional application No. 63/048,634, filed on Jul. 6, 2020.

(51) Int. Cl. A61L 9/16; F24F 3/14; F24F 1/0328

(52) U.S. Cl. CPC A61L 9/16; F24F 3/1417; F24F 1/0328; A61L 2209/14; A61L 2209/16

(57) ABSTRACT

A system and method reduce a concentration of viable microorganisms, including but not limited to the 2019 novel (new) coronavirus that causes the illness coronavirus disease 2019 (COVID-19), in air in a workspace by over 80% with a single pass of air through the system. A heater heats air drawn from the workspace and a desiccant dehumidifier further heats air from the heater. The heater and desiccant dehumidifier decontaminate the air to kill or inactivate the microorganisms so that air returned to the workspace from the system has a reduced concentration of viable microorganisms.

### RELATED PATENTS AND APPLICATIONS

This disclosure relates to the inventor's prior U.S. Pat. Nos. 6,652,628; 7,022,167; 7,284,383; 7,284,384; 7,284,385; 7,284,386; 7,284,387; 7,789,937; and 8,361,206, the contents of all of which are hereby incorporated by reference herein, in their entirety. These prior patents primarily relate to desiccant dehumidifiers and applications thereof, and as set forth in more detail below, the system and method disclosed herein utilize desiccant dehumidifiers.

### FIELD OF THE DISCLOSURE

The disclosure relates to a system and method for killing microorganisms. One aspect of the disclosure relates to a system and method operating as an “air autoclave”, decontaminating at high temperature.

### BACKGROUND

To date, our general understanding is that the 2019 novel (new) coronavirus that causes the illness coronavirus disease 2019 (COVID-19) can be spread through the air and is suspected to remain airborne as a viable contaminant for prolonged periods (hours, possibly days). Thus, there is a great risk of transmittal and spread. The concern is particularly acute in enclosed spaces.

While some existing air handling and purifying systems may have some efficacy in terms of killing microorganisms, the inventor is not aware of any system that mitigates indoor environments to an extent that minimizes, if not eliminates, the risk of transmittal and spread of COVID-19.

Thus, there exists a need for an air handling system and method that treats air so as to accomplish a substantial reduction in the concentration of air-borne microorganisms such as the 2019 novel coronavirus, where the treatment is accomplished without exposing the air to toxic chemicals.

### SUMMARY OF THE DISCLOSURE

One aspect of the disclosure relates to a system for reducing a concentration of viable microorganisms, including but not limited to the 2019 novel (new) coronavirus that causes the illness coronavirus disease 2019 (COVID-19), in air in a workspace by over 80% with a single pass of air through the system. The system comprises a heater for heating air drawn from the workspace and a desiccant dehumidifier for further heating air from the heater. The heater and desiccant dehumidifier decontaminate the air to kill or inactivate the microorganisms so that air returned to the workspace from the system has a reduced concentration of viable microorganisms.

Depending on the application, the heater heats air drawn from the workspace to at least about 130° F. (54.4° C.) and/or desiccant in the desiccant dehumidifier is heated to about 270° F. (132.2° C.) to about 500° F. (260° C.).

In an embodiment, air enters the heater from a return located in the workspace. The workspace can include at least one air circulator to direct air towards the return. The at least one air circulator can include filtering to trap at least some airborne particles. The filtering can include a HEPA filter.

The system can further comprise a valving/diverter arrangement to recirculate air through at least one of the heater and desiccant dehumidifier. Additionally, the system can also include an air conditioning unit for cooling the air prior to return to the workspace. In exemplary embodiments, the workspace and/or the system and/or one of the system components includes at least one germicidal lamp.

Another aspect of the disclosure relates to a method for reducing a concentration of viable microorganisms, including but not limited to the 2019 novel (new) coronavirus that causes the illness coronavirus disease 2019 (COVID-19), in air in a workspace by over 80% with a single pass of air through a system including a heater and a desiccant dehumidifier. The method comprises heating air drawn from the workspace in the heater; and further heating air from the heater in the desiccant dehumidifier. The heating and further heating decontaminate the air to kill or inactivate the microorganisms so that air returned to the workspace from the system has a reduced concentration of viable microorganisms.

Depending on the application, the heater heats air drawn from the workspace to at least about 130° F. (54.4° C.) and/or desiccant in the desiccant dehumidifier is heated to about 270° F. (132.2° C.) to about 500° F. (260° C.).

In an embodiment, air enters the heater from a return located in the workspace. The workspace can include at least one air circulator to direct air towards the return. The at least one air circulator can include filtering to trap at least some airborne particles. The filtering can include a HEPA filter.

The system can further comprise a valving/diverter arrangement to recirculate air through at least one of the heater and desiccant dehumidifier. Additionally, the system can also include an air conditioning unit for cooling the air prior to return to the workspace.

In exemplary embodiments, the workspace and/or the system and/or one of the system components includes at least one germicidal lamp. In this regard, the at least one germicidal lamp can be provided in at least one of the heater, desiccant dehumidifier, air conditioning unit, and connecting ductwork.

### BRIEF DESCRIPTION OF THE DRAWINGS

A more complete understanding of the present disclosure, and the attendant advantages and features thereof, will be more readily understood by reference to the following description when considered in conjunction with the accompanying drawings wherein:

FIG. 1 shows a schematic representation of an embodiment of the system according to the disclosure.

FIG. 2 shows a schematic representation in which a single system is used for entire building or portion thereof.

FIG. 3 shows a schematic representation in which different rooms or portions of a building have their own system.

### DETAILED DESCRIPTION

As required, embodiments are disclosed herein; however, it is to be understood that the disclosed embodiments are merely examples and that the methods described below can be embodied in various forms. Therefore, specific structural and functional details disclosed herein are not to be interpreted as limiting, but merely as a representative basis for teaching one skilled in the art to variously employ the present subject matter in virtually any appropriately detailed structure and function. Further, the terms and phrases used herein are not intended to be limiting, but rather, to provide an understandable description of the concepts.

It can be advantageous to set forth definitions of certain words and phrases used throughout this disclosure. The terms “a” or “an”, as used herein, are employed to describe elements and components described herein. This is done merely for convenience and to give a general sense of the scope of the disclosure. This description should be read to include one or at least one and the singular also includes the plural unless it is obvious that it is meant otherwise. The term plurality, as used herein, is defined as two or more than two. The term another, as used herein, is defined as at least a second or more. The term “coupled,” as used herein, is defined as “connected,” although not necessarily directly, and not necessarily mechanically.

The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, and C; A and B; A and C; B and C; A alone; B alone; and C alone.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Note that not all of the activities described above in the general description or the examples are required, that a portion of a specific activity may not be required, and that one or more further activities can be performed in addition to those described. Still further, the order in which activities are listed are not necessarily the order in which they are performed.

In general, the disclosure relates to mitigating indoor environments to an extent that minimizes, if not eliminates, the risk of transmittal and spread of COVID-19 and any other airborne virus or microorganism by cycling air through a system. This system has been named the “Viro Kill Air-wash System”, or VKAS. The following details the system, including its parts, layout and its operation.

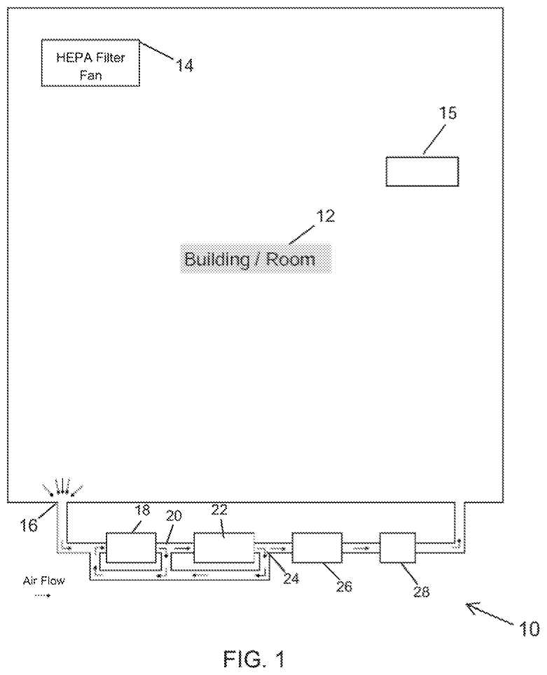

Referring to FIG. 1, a schematic representation of an embodiment of a system 10 according to the disclosure is shown. System 10 continuously disinfects and recirculates indoor air within workspace 12. As used herein, the term “disinfects” and derivatives thereof mean treatment of the air so that aerosolized microorganisms are inactivated or killed to an extent that minimizes, if not eliminates, airborne transmission of active viable microorganisms. The treatment of the air depends on a number of factors, including the size of the room, the initial concentration of the microorganisms, etc. Experimental work has found that system 10 can inactivate or kill over 90% of microorganisms in a single pass. The disclosure contemplates that inactivation or killing of 80% or more of microorganisms minimizes, if not eliminates, airborne transmission of active viable microorganisms.

As set forth in more detail below, workspace 12 can be an entire building or a portion of the building, including but not limited to one or more floors of a building and/or one or more rooms or enclosed spaces within the building. Although FIG. 1 shows most components of system 10 as external to the workspace 12, the disclosure contemplates that other components can be located inside the workspace 12.

In one embodiment, system 10 is operated to result in one air change of workspace 12 per hour. However, in other applications, such as areas in hospitals or other healthcare facilities, system 10 can be operated to result in two or more air change of workspace 12 per hour.

Air from workspace 12, which is preferably under negative pressure, is pulled into a linear chain of equipment that comprises system 10. In an exemplary embodiment, one or more fans 14 or other air circulators, optionally with a filtering system such as HEPA filter(s), are located in workspace 12 to direct air towards return 16 as well as initially remove at least some airborne particles and/or other contaminants. The disclosure also contemplates the use of electric jet power blowers to assist in moving air in workspace 12 from all surfaces, included those in confined locations.

The disclosure further contemplates that one or more germicidal lamps 15 can be placed in workspace 12. For example, ultraviolet radiation can be used for disinfection. UV wavelengths are in the range of 200 nm to 390 nm and have optimal UV germicidal action at 254-265 nm. For UV sterilization, only UV-C (100-280 nm) has high enough energy to effectively kill microorganisms. Although UV-C can immediately kill pathogens, UV-C generally should be used without humans present (or the exposure of humans should limited to no more than a certain number of minutes per day) because of potential adverse health consequences.

UV-A (315 to 400 nm band) energy or short-wavelength energy in the violet or blue range destroy pathogens over longer exposure times. The typical product mixes violet LEDs and white LEDs in a luminaire and only uses the violet LEDs during periods when a space is being disinfected. The mixed white and violet light is still usable for humans working in the space. The violet energy destroys pathogens over long exposure times.

Although there is evidence that FAR UV-C (207-222 nm) is considered both safe to humans and effective in killing microorganisms, FAR UV-C does not have the extensive data supporting the safety and efficacy that UV-A has.

After air enters return 16, the air travels to a heater 18. In a non-limiting example, the air flow can be up to around 1200 feet per minute and external static pressure can be more than 3 inches. Heater 18 heats the air to a desired temperature. For COVID-19, the desired temperature is a minimum of about 130° F. (54.4° C.). In testing systems, the temperature has been measured as high as approximately 450° F. (232.2° C.). A first valving/diverter arrangement 20 can recirculate air through heater 18 until a set temperature and/or time is reached.

After being heated, the air is drawn into a desiccant dehumidifier 22. Desiccant dehumidifier 22, like the desiccant dehumidifiers disclosed in the inventor's above-identified related patents, uses an internal heater to regenerate the desiccant. Additionally, the heat of the desiccant is such that the heat kills and/or inactivates contaminants, including but not limited to bacteria and viruses. Accordingly, the temperature can be any suitable temperature range that effectively achieves the killing and/or inactivation. In testing system, the internal heat of the desiccant has been measured as approximately 270° F. (132.2° C.) to approximately 500° F. (260° C.). The disclosure contemplates that the temperature air is heated through heater 18 and/or desiccant dehumidifier 22 can be selected not only to achieve the desired killing and/or inactivation, but also to account for conditions of workspace 12.

It should also be noted that desiccant dehumidifier 22, again like any desiccant dehumidifier, uses a desiccant in a wheel through which the air is pulled into the dehumidifying chamber. The high velocity of the air, in addition to the heat, can also contribute to the killing effect by mechanically disrupting microorganisms upon contact with the wheel. In this regard, the wheel can be made of and/or treated (e.g. coated) with a material (e.g. copper) that has antimicrobial properties. Cooper is also particularly useful since it is a material with high heat conductivity.

As the air passes through desiccant dehumidifier 22, it can be recirculated back either to heater 18 or desiccant dehumidifier 22 in order to complete the process again to ensure the inactivation of all contaminants. A second valving/diverter arrangement 24 can control the re-circulation until the desired number of cycles is reached. The desired number of cycles can be determined empirically, analytical, or any other suitable manner.

After the re-circulation, the air will move through an air conditioning unit 26 for cooling and, optionally, an air machine 28 with HEPA filtration that will allow the cleaned air to return to workspace 12. For example, if workspace 12 is occupied, air conditioning unit 26 can be set to lower temperatures compared to those used if workspace 12 is unoccupied. Additionally, system 10, like workspace 12, can be provided with germicidal lamps to supplement the killing effect of system 10. These germicidal lamps can be located in one or more of the components of system 10 and/or in the ductwork connect the components of system 10.

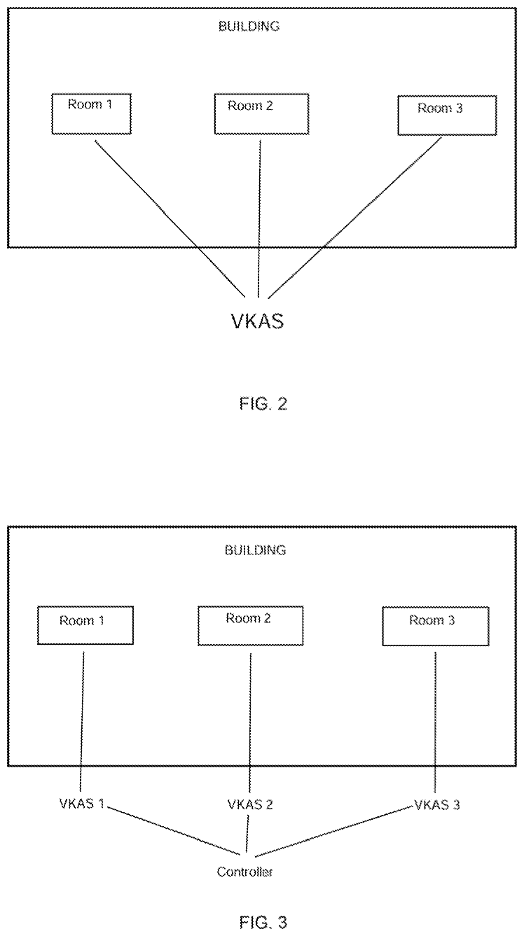

In the embodiment shown in FIG. 2, a single VKAS system is used to treat multiple workspaces in a building. In the embodiment shown in FIG. 3, each workspace is treated with its individual VKAS system so that a controller can control the treatment of each workspace individually.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the disclosure, skilled artisans will appreciate that certain features are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A system for reducing a concentration of viable microorganisms, including but not limited to the 2019 novel (new) coronavirus that causes the illness coronavirus disease 2019 (COVID-19), in air in a workspace by over 80% with a single pass of air through the system, the system comprising:
a heater for heating air drawn from the workspace; and
a desiccant dehumidifier for further heating air from the heater,
wherein the heater and desiccant dehumidifier decontaminate the air to kill or inactivate the microorganisms so that air returned to the workspace from the system has a reduced concentration of viable microorganisms.

2. The system of claim 1, wherein air enters the heater from a return located in the workspace and wherein the workspace includes at least one air circulator to direct air towards the return.

3. The system of claim 2, wherein the at least one air circulator includes filtering to trap at least some airborne particles.

4. The system of claim 3, wherein the filtering includes a HEPA filter.

5. The system of claim 1, further comprising a valving/diverter arrangement to recirculate air through at least one of the heater and desiccant dehumidifier.

6. The system of claim 1, further comprising an air conditioning unit for cooling the air prior to return to the workspace.

7. The system of claim 1, wherein the workspace includes at least one germicidal lamp.

8. The system of claim 1, further comprising at least one germicidal lamp.

9. The system of claim 1, wherein the heater heats air drawn from the workspace to at least about 130° F. (54.4° C.).

10. The system of claim 1, wherein desiccant in the desiccant dehumidifier is heated to about 270° F. (132.2° C.) to about 500° F. (260° C.).

11. A method for reducing a concentration of viable microorganisms, including but not limited to the 2019 novel (new) coronavirus that causes the illness coronavirus disease 2019 (COVID-19), in air in a workspace by over 80% with a single pass of air through a system including a heater and a desiccant dehumidifier, the method comprising:
heating air drawn from the workspace in the heater; and
further heating air from the heater in the desiccant dehumidifier,
wherein the heating and further heating decontaminate the air to kill or inactivate the microorganisms so that air returned to the workspace from the system has a reduced concentration of viable microorganisms.

12. The method of claim 11, wherein air enters the heater from a return located in the workspace and wherein the workspace includes at least one air circulator to direct air towards the return.

13. The method of claim 12, wherein the at least one air circulator includes filtering to trap at least some airborne particles.

14. The method of claim 13, wherein the filtering includes a HEPA filter.

15. The method of claim 11, wherein a valving/diverter arrangement recirculates air through at least one of the heater and desiccant dehumidifier.

16. The method of claim 11, wherein an air conditioning unit cools the air prior to return to the workspace.

17. The method of claim 11, wherein the workspace includes at least one germicidal lamp.

18. The method of claim 11, wherein at least one germicidal lamp is provided in at least one of the heater, desiccant dehumidifier, air conditioning unit, and connecting ductwork.

19. The method of claim 11, wherein the heater heats air drawn from the workspace to at least about 130° F. (54.4° C.).

20. The method of claim 11, wherein desiccant in the desiccant dehumidifier is heated to about 270° F. (132.2° C.) to about 500° F. (260° C.).
